(19) United States
(12) Patent Application Publication
(10) Pub. No.: US 2002/0086428 A1
(43) Pub. Date: Jul. 4, 2002

(54) Methods and compositions for independent DNA replication in eukaryotic cells

(75) Inventors: John Jelesko, Blacksburg, VA (US); Wilhelm Gruissem, Zuerich (CH)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, Office of Technology transfer, Oakland, CA (US)

(21) Appl. No.: 09/944,715

(22) Filed: Aug. 31, 2001

Related U.S. Application Data
(60) Provisional application No. 60/229,686, filed on Sep. 1, 2000.

(51) Int. Cl. C12N 15/86; C12N 15/85

(52) U.S. Cl. 435/455; 435/456; 435/468

(57) ABSTRACT

The invention provides methods and compositions for replicating a polynucleotide sequence independent of replication of chromosomal DNA in a cell.

### CROSS REFERENCE OF RELATED PATENT APPLICSTIONS

The present application benefit of priority to U.S. Provisional Patent Application No. 60/229,686, filed Sep. 1, 2000, which is explicitly incorporated herein reference in its entirety and for all purposes.

### STATEMENT AS TO RIGHTS TO INVENTIONS MADE UNDER FEDERALLY SPONSORED RESEARCH AND DEVELOPMENT

This invention was made with Government support under National Science Foundation grants INT-9622319, INT-9743534 and IBN-9727044. The Government has certain rights in this invention.

### FIELD OF THE INVENTION

This invention relates generally to methods and compositions for replicating a polynucleotide sequence independent of replication of chromosomal DNA in a cell.

### BACKGROUND OF THE INVENTION

DNA replication has been studied for many years in both prokaryotic and eukaryotic cells. See, e.g. Kornberg, DNA REPLICATION (W. H. Freeman & Co., 1980); Alberts, Philos. Trans. R. Soc. Lond. B. Biol. Sci. 317(1187):395-420 (1987). Early studies of DNA replication focused on native E. coli DNA replication mechanisms, but were soon followed by studies of phage DNA replication in E. coli. Among the phage studied were T4, T5 and T7.

The enzyme that replicates the T7 phage genome is comprised of two parts: the 80 kdal T7 DNA polymerase (T7 gene 5 protein) and the 12 kdal thioredoxin protein of E. coli. The T7 polymerase complex has both 5′→3′ polymerase activity and 3′→5′ exonuclease activity.

Initiation of T7 DNA replication by the T7 DNA polymerase complex requires that T7 RNA polymerase bind to the T7 promoter and synthesize a short RNA transcript. The transcript functions as a primer and is extended by the DNA polymerase complex, which travels along the template DNA and synthesizes a complementary leading strand of replicated DNA in the 5′→3′ direction.

Since the DNA polymerase only synthesizes DNA in the 5′→3′ direction, the lagging strand of DNA is synthesized in short sections that are subsequently ligated together. For synthesis of these short DNA sections by the DNA polymerase, primers are first synthesized by the 58 kdal T7 gene 4 protein. This protein functions both as a DNA helicase and a primase. The short RNA transcripts formed by the primase activity (generally pppACCA or pppACCC, referred to as Okazaki fragments) are subsequently extended by the DNA polymerase.

Of the enzymes discussed above, only T7 RNA polymerase has been expressed in eukaryotic cells. See, Benton, et al., Mol. Cell. Biol. 10(1):353-60 (1990) and Dunn et al., Gene 68(2):259-66 (1988). The goal of these studies, however, was to initiate T7-based RNA transcription initiated from a T7 promoter in eukaryotes, not to initiate DNA replication. To date, there have been no known reports of a prokaryotic DNA replication system in eukaryotes. The present invention addresses these and other problems.

### SUMMARY OF THE INVENTION

This invention provides methods of regulating the replication of a DNA molecule independent of endogenous chromosomal or plasmid replication. In some embodiments, the methods comprise (1) introducing into a eukaryotic cell, a replication cassette comprising an origin of replication and introducing a replication system. The replication system comprises a polynucleotide encoding a polypeptide with RNA polymerase activity, a polynucleotide encoding a polypeptide with DNA polymerase activity, a polynucleotide encoding a polypeptide with DNA helicase activity and a polynucleotide encoding a polypeptide with DNA primase activity. In some embodiments, the replication system further comprises a single-stranded DNA binding polypeptide. Moreover, the polynucleotide encoding each polypeptide is operably linked to a eukaryotic replication promoter. The methods of the invention thereby initiate replication of the replication cassette independent from chromosomal DNA replication. In some embodiments, the methods of the invention provide for an increase in the number of copies of the replication cassette.

The invention also provides for a eukaryotic organism comprising a polynucleotide encoding each of the following polypeptides: T7 RNA polymerase, T7 gene 4 protein, T7 DNA polymerase and TrxA, wherein the polynucleotide encoding each polypeptide is operably linked to a promoter. In some embodiments, the eukaryotic organism comprises a polynucleotide encoding a single-stranded DNA binding protein. The organism can be, for instance, a plant, including a plant cell in a plant cell culture. In some embodiments, the replication cassette is episomal. For example, the replication cassette can be an episomal plasmid. Alternatively, the replication cassette can be integrated into a eukaryotic chromosome.

The invention further provides a polynucleotide comprising a bacteriophage T7 origin of replication, a recombination sequence and an expression cassette comprising a eukaryotic replication promoter. In some embodiments, the polynucleotide comprises a T7 promoter.

In some embodiments, the replication system comprises a polynucleotide encoding each of the following polypeptides: T7 RNA polymerase, T7 gene 4 protein, T7 DNA polymerase and TrxA. In some embodiments, the replication system comprises a single-stranded DNA binding protein. Any or all of the replication system polynucleotides can additionally encode a nuclear localization signal sequence. For example, at least one, or all, of the following polynucleotides can encode a nuclear localization signal: T7 RNA polymerase, T7 gene 4 protein, T7 DNA polymerase, TrxA and the single-stranded DNA binding protein, such as the T7 2.5 gene product.

The eukaryotic cell can be, for instance, a plant cell or a mammalian cell. The origin of replication can be, for instance, a T7 bacteriophage origin of replication.

The replication cassette can comprise a T7 promoter and/or an expression cassette. The expression cassette can comprise a polynucleotide operably linked to an expression promoter in the antisense or sense orientation. The replication cassette can also comprise at least 200 base pairs of DNA that is at least 70% identical to chromosomal DNA in the eukaryotic cell. For example, the replication cassette can comprise at least 200 base pairs of DNA that is identical to chromosomal DNA in the eukaryotic cell.

In some embodiments, the replication cassette comprises a recombination sequence such as a lox sequence. The replication system polynucleotides can also further encode a sequence-specific recombinase such as the Cre recombinase.

The eukaryotic replication or expression promoters can be constitutive and/or tissue-specific. The eukaryotic replication promoter can be, for instance, a meiosis-specific promoter. In some embodiments, the eukaryotic replication promoter is inducible.

### DEFINITIONS

The term “T7 origin of replication” refers to the phi OR promoter (Genbank Accession number V01146) starting at position 39,229, or other sequence sufficient for initiation of DNA replication by T7 DNA polymerase. One of skill in the art can identify variants of the phi OR promoter to initiate DNA replication by T7 DNA polymerase.

The term “replication cassette” refers to a polynucleotide sequence that can be replicated in a eukaryotic cell independent of chromosomal DNA replication.

“Chromosomal DNA replication” refers to the replication of the nuclear chromosome of a cell as prescribed by the cell cycle. Replication typically occurs in a regulated fashion during mitosis.

“DNA polymerase” refers to a polypeptide with DNA polymerase activity, i.e., the enzymatic activity of replicating a DNA molecule. DNA replication comprises synthesis of two complementary single stranded DNA molecules, each of which are synthesized as a complement to a template DNA molecule. The single stranded DNA molecules are typically synthesized in the 5′→3′ direction. A DNA polymerase can also have additional enzymatic activities, including, for example, DNA helicase and primase activity. Polypeptides with DNA polymerase activity include prokaryotic DNA polymerases such as the T4, T5 and T7 DNA polymerases, E. coli DNA polymerase I, II and III, Taq polymerase, as well as eukaryotic polymerases such as plant and animal DNA polymerases α, β and γ. See, e.g., Kornberg, supra; Burgers, Chromosoma 107(4):218-27 (1998); Hubscher et al, Trends Biochem Sci 25(3):143-7 (2000).

A “primase” refers to a polypeptide with primase activity, i.e., the ability to synthesize small RNA or DNA oligonucleotide primers. The primers provide an oligonucleotide that will bind the DNA template and provide at least one initial nucleotide from which the DNA polymerase can catalyze the addition of nucleotides complementary to the DNA template. Primases can also have additional enzymatic activities, including, for example, DNA helicase and polymerase activity. Polypeptides with primase activity include prokaryotic pimases such as the E. coli primase (dnaG protein), gene 4 protein of phage T7 , Gene 41 and 61 of phage T4. See, e.g., Kornberg, supra.

“DNA helicase” refers to a polypeptide with helicase activity, i.e., the unwinding of supercoiled DNA thereby allowing for clear access of the DNA polymerase to the DNA template. Helicases can also have additional enzymatic activities, including, for example, primase and polymerase activity. Polypeptides with primase activity include E. coli helicase I, phage T7 gene 4 protein, and S. cervisae helicases A-D. See, e.g., Matson, et al., J Biol Chem 266(24):16232-7 (1991); Frei, et al., J Cell Sci 2000; 113(Pt 15):2641-2646; Li, et al., Chromosoma 102(1 Suppl):S93-9 (1992).

“RNA polymerase” refers to a polypeptide with RNA polymerase activity, i.e., the ability to synthesize an RNA molecule complementary to a DNA template.

A “single-stranded DNA binding polypeptide” refers to a polypeptide with an affinity for single-stranded DNA. Thus, the polypeptide coats single-stranded DNA molecules, thereby removing secondary structure in the DNA molecule. These polypeptides can also interact with other DNA associated polypeptides, thereby enhancing DNA replication and recombination processes. Polypeptides with single-stranded DNA binding activity include the T7 gene 2.5 protein, E. coli SSB protein, T4 gene 32 protein, SSB proteins from Xenopus, Saccharomyces cerevisiae, human mitochondria and adenoviruses. See, e.g., Hollis, T., et al. Proc. Natl. Acad. Sci. USA, 98(17) 9557-9562 (2001); Komberg, supra.

A “recombination sequence” refers to a polynucleotide sequence that is recognized and cleaved by a sequence-specific recombinase. For example, a lox site is is recombination sequence corresponding to the Cre recombinase.

An “episome” is a polynucleotide that is physically separated from the chromosomes of a cell. For example, plasmids are episomes.

A “sequence-specific recombination site” refers to a specific sequence that is recognized by a sequence-specific recombinase, thereby resulting in the cleavage and re-ligation of the site to the same or new recombination site. Examples of recombinases and their respective sequence-specific recombination sites include the Crellox and FLP/FRT systems described in detail below.

The phrase “nucleic acid sequence” refers to a single or double-stranded polymer of deoxyribonucleotide or ribonucleotide bases read from the 5′ to the 3′ end. It includes chromosomal DNA, self-replicating plasmids, infectious polymers of DNA or RNA and DNA or RNA that performs a primarily structural role.

A “promoter” is defined as an array of nucleic acid control sequences that direct transcription of an operably linked nucleic acid. A “eukaryotic promoter” refers to any promoter capable of controlling or initiating transcription of an operably linked polynucleotide in a eukaryotic cell. As used herein, a “plant promoter” is a promoter that functions in plants. Promoters include necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, plant organs (e.g., leaves, stems, flowers, roots, undifferentiated cell cultures, etc.), seeds and plant cells and progeny of same. The class of plants which can be used in the method of the invention is generally as broad as the class of flowering plants amenable to transformation techniques, including angiosperms (monocotyledonous and dicotyledonous plants), as well as gymnosperms. It includes plants of a variety of ploidy levels, including polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally occurring allelic variants.

A polynucleotide “exogenous to” an individual plant is a polynucleotide which is introduced into the plant, or a predecessor generation of the plant, by any means other than by a sexual cross. Examples of means by which this can be accomplished are described below, and include Agrobacterium-mediated transformation, biolistic methods, electroporation, in planta techniques, and the like.

An “expression cassette” refers to a polynucleotide with a series of nucleic acid elements that permit transcription of a particular nucleic acid in a cell. Typically, the expression cassette includes a nucleic acid to be transcribed operably linked to a promoter.

The phrase “host cell” refers to a cell from any organism. Preferred host cells are derived from plants, bacteria, yeast, fungi, insects or other animals. Methods for introducing polynucleotide sequences into various types of host cells are well known in the art.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA).

The phrase “substantially identical,” in the context of two nucleic acids or polypeptides, refers to a sequence or subsequence that has at least 40% sequence identity with a reference sequence. Alternatively, percent identity can be any integer from 40% to 100% (e.g., 40%, 41%, 42%, etc.). More preferred embodiments include at least: 40%, 45%, 50%, 55%, 60%, 65%, 70%, 75%, 80%, 85%, 90%, 95%, or 99%. compared to a reference sequence using the programs described herein; preferably BLAST using standard parameters, as described below. This definition also refers to the complement of a test sequence, when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a usefull algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W). (see, e.g., Creighton, Proteins (1984)).

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 time background hybridization.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cased, the nucleic acids typically hybridize under moderately stringent hybridization conditions.

In the present invention, genomic DNA or cDNA comprising nucleic acids of the invention can be identified in standard Southern blots under stringent conditions using the nucleic acid sequences disclosed here. For the purposes of this disclosure, suitable stringent conditions for such hybridizations are those which include a hybridization in a buffer of 40% fornamide, 1 M NaCl, 1% SDS at 37° C., and at least one wash in 0.2X SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

### BRIEF DESCRIPTION OF THE DRAWINGS

FIG. 1 illustrates the cloning strategy for constructing a polynucleotide encoding T7 DNA polymerase (gene 5).

FIG. 2 illustrates the cloning of the E. Coli gene TrxA.

FIG. 3 illustrates the cloning strategy for constructing a polynucleotide encoding T7 primase/helicase (gene 4A′).

FIGS. 4 and 5 illustrate the cloning strategy for constructing a polynucleotide encoding T7 RNA polymerase. FIG. 5 illustrates correcting the 3′ end of pJGJ310 and 312.

FIG. 6 illustrates a general strategy for subcloning the replication system polynucleotides into plasmid vectors.

FIG. 7 is a schematic representation of an embodiment of the invention. The Figure illustrates initiation of DNA replication and processing of the resulting replicated DNA into circular units.

### DETAILED DESCRIPTION

This invention provides methods and compositions useful for regulating the replication of a DNA cassette. Specifically, the invention provides a replication system capable of initiating replication of a DNA replication cassette, the system comprising cis and trans acting factors capable of initiating DNA replication from an origin of replication within a replication cassette. For example, transgenic eukaryotic cells expressing bacteriophage T7 trans- and cis-acting proteins can be used to replicate a polynucleotide replication cassette comprising a T7 origin of replication.

The invention provides methods for inducing replication of the replication cassette independent of the replication of chromosomal DNA. Regulation of expression of the replication system (e.g., T7 and E. coli trans and cis-acting factors) allows for control of replication of the replication cassette. Moreover, the quantity of replication can be regulated by controlling the expression of one or more components of the replication system. Thus, increased expression of the trans- and cis-acting proteins induce replication of the replication cassette and leads to the production of episomal DNA constructs within a cell's nucleus. This invention is useful in any circumstances where an increase in gene copy number or decrease in gene expression (e.g., antisense, sense suppression, etc.) is desired.

The present invention provides methods of facilitating homologous recombination events of a transgene on the replication cassette with the endogenous, chromosomal gene. Without intending to limit the invention, it is believed that increasing the copy number of a transgene of choice will increase the probability that a recombination event will occur.

The present invention also provides for modulating transformation and expression of a transgene on a replication cassette by modulating the copy number of the replication cassette.

### A. Compositions of the Invention

1. Replication System Polynucleotides

The polynucleotides of the invention comprise coding sequences for a set of polypeptides (the replication system) capable of inducing replication from a specific polynucleotide sequence. Typically, the polypeptides that act to replicate the polynucleotides will comprise prokaryotic or viral cis and trans-acting polypeptides that initiate DNA replication. In some preferred embodiments, the polypeptides of the replication system have the following activities: DNA polymerase activity, primase activity or helicase activity. In some embodiments, the replication system polypeptides comprise T7 DNA polymerase, T7 helicase/primase, and E. coli thioredoxin genes. In some embodiments, the replication system further comprises a single-stranded DNA binding protein.

DNA polymerase activity refers to the enzymatic activity of replicating a DNA molecule. DNA replication comprises synthesis of a two complementary single stranded DNA molecules, each of which are synthesized as a complement to a template DNA molecule. The single stranded DNA molecules are typically synthesized in the 5′→3′, direction. Polypeptides with DNA polymerase activity include prokaryotic DNA polymerases such as the T4, T5 and T7 DNA polymerases, E. coli DNA polymerase I, II and III, Taq polymerase, as well as eukaryotic polymerases such as animal DNA polymerases α, β, and γ. See, e.g., Komberg, supra.

Primase activity refers to synthesis of small RNA or DNA oligonucleotide primers. The primers provide an oligonucleotide that will bind the DNA template and provide at least one initial nucleotide from which the DNA polymerase can catalyze the addition of nucleotides complementary to the DNA template. Polypeptides with DNA polymerase activity include prokaryotic DNA polymerases such as the T4, T5 and T7 DNA polymerases, E. coli DNA polymerase I, II and III, Taq polymerase, as well as eukaryotic polymerases such as plant and animal DNA polymerases α, β and γ. See, e.g., Komberg, supra; Burgers, Chromosoma 107(4):218-27 (1998); Hubscher et al., Trends Biochem Sci 25(3):143-7 (2000).

DNA helicase activity refers to the unwinding of supercoiled DNA thereby allowing for clear access of the DNA polymerase to the DNA template. Polypeptides with primase activity include E. coli helicase I, phage T7 gene 4 protein, and S. cervisae helicases A-D. See, e.g., Matson, et al., J Biol Chem 266(24):16232-7 (1991); Frei, et al., J Cell Sci 2000; 113(Pt 15):2641-2646; Li, et al., Chromosoma 102(1 Suppl):S93-9 (1992).

In addition, the replication system can further include polypeptides with RNA polymerase activity. RNA polymerase activity refers to the synthesis of an RNA molecule complementary to a DNA template. For example, in some embodiments, the replication system comprises T7 RNA polymerase, or a mutant version that has a tendency to synthesize short mRNA transcripts, e.g., the A408T mutation. See, e.g., Lyakhov, et al, J. Mol. Biol. 269(1): 28-40 (1997) and Zhang, et al., J. Mol. Biol. 250(2):156-168 (1995).

In some embodiments, the replication system further comprises a single-stranded DNA binding polypeptide. Such polypeptides are useful to enhance DNA replication processes. Typically, single-stranded DNA binding proteins bind singlestranded DNA independent of sequence. Exemplary single-stranded DNA binding proteins include the 2.5 gene protein from T7 phage.

The nucleotide sequence encoding phage T7 proteins can be found at Genbank Accession No. V01146 J02518 X00411, which comprises the entire annotated genome of phage T7.

2. Nuclear Localization Signal Sequences

To replicate DNA in eukaryotic cells, the polypeptides regulating replication typically function in the nucleus of a cell. Therefore, in some preferred embodiments of the invention, the polypeptides of the replication system comprise a nuclear localization signal sequence. In preferred embodiments, the nuclear localization signal sequence is attached to the amino or carboxy terminus of the polypeptides. Alternatively, the signal sequence can be inserted within the primary structure of the polypeptide. Preferably, the nuclear localization signal sequence and polypeptide are synthesized as a fusion protein.

Nuclear localization signal sequences are known to those of ordinary skill in the art. See, e.g., Nakielny, et al., Cell 99:677-690 (1999). Generally, nuclear localization signal sequences are from four to eight amino acids in length and are positively charged. Nuclear localization signal sequences typically include lysine, arginine and/or proline residues. For example, one preferred nuclear localization signal sequence is PKKKRKV. In some embodiments, the nuclear localization signal sequence is comprised of at least two subsequences, each four amino acids long, that are separated by approximately 10 amino acids. A preferred nuclear localization signal sequence is the 36 base pair SV40 T antigen nuclear location signal sequence. See, e.g., Benton, et al., Mol. Cell. Biol. 10(1):353-60 (1990) and Dunn, et al., Gene 68(2):259-66 (1988).

3. Mechanism of Replication

The replication system functions by adding a new DNA replication system to a cell. Typically, the replication system is based on a prokaryotic or viral (including phage) DNA replication system. The replication is under the control of a heterologous promoter that allows for the control of the replication system independent of the cells chromosomal DNA replication machinery.

As described above, the mechanisms of DNA replication are well known. A brief summary of DNA replication is summarized below. First, an RNA polymerase synthesizes short RNA transcripts at or near the origin of replication. This short RNA remains bound to the DNA and provides a primer for a DNA polymerase (e.g, the T7 DNA polymerase-thioredoxin complex). The DNA polymerase complex synthesizes the leading strand of single-stranded DNA. The lagging strand is initiated by synthesis of short DNA transcripts via a primase/helicase enzyme(s). This short transcript is then extended by the polymerase complex. As a result, both DNA strands of the replication cassette will be replicated. See, generally, Darnell, et al., MOLECULAR CELL BIOLOGY (Scientific American Books, 1986), Chapter 13 and Alberts et al., MOLECULAR BIOLOGY OF THE CELL (Garland Publishing, 1994), pages 251-262.

In some embodiments, DNA replication is enhanced by providing a single-stranded DNA binding protein such as the T7 2.5 gene product.

In embodiments where the original template is circular, the replication reaction will form concatamers as result of repeated DNA replication around the circle of DNA. Concatamer formation is analogous to a rolling circle model of replication. In embodiments where a sequence-specific recombination site is present on the replication cassette, the linear concatamers are processed by a recombinase, as described below, into individual circular DNA molecules.

4. Control of the Time and Amount of Replication

The time and amount of DNA replication of the replication cassette depends on the expression of the polypeptides of the replication system. By controlling expression of the replication system polypeptides, replication of the replication cassette can be tightly controlled.

Controlling the time when, or tissue where, the replication system is expressed controls when, or where, the replication cassette is replicated. The replication promoters of the invention regulate this activity. For example, as discussed below, a meiosis-specific promoter can be operably linked to a polynucleotide encoding the replication-specific polypeptides to induce replication in cells to improve the probability of homologous recombination.

Alternatively, the replication system of the invention can be used to control expression of a gene of interest on the replication cassette, i.e. the “expression promoter.” Increased copy number of a polynucleotide comprising an expression cassette can lead to increased transcription from the cassette. Thus, increased expression of the replication-specific polypeptides leads to increased expression of the genes residing on the replication cassette.

a. Constititutive promoters

In some embodiments, a promoter fragment is employed which directs expression of the genes in all tissues of an organism. Such promoters are referred to herein as “constitutive” promoters and are active under most environmental conditions and states of development or cell differentiation. Examples of constitutive plant promoters include the cauliflower mosaic virus (CaMV) 35S transcription initiation region, the 1′- or 2′- promoter derived from T-DNA of Agrobacterium tumafaciens, and other transcription initiation regions from various plant genes known to those of skill. Such genes include for example, ACT11 from Arabidopsis (Huang et al. Plant Mol. Biol. 33:125-139 (1996)), Cat3 from Arabidopsis (GenBank No. U43147, Zhong et al., Mol. Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), and Gpc2 from maize (GenBank No. U45855, Manjunath et al., Plant Mol. Biol. 33:97-112 (1997)).

Examples of mammalian promoters include CMV promoter, SV40 early promoter, SV40 late promoter, metallothionein promoter, murine mammary tumor virus promoter, Rous sarcoma virus promoter, polyhedrin promoter, or other promoters shown effective for expression in animal cells.

b. Tissue-Specific Promoters

Alternatively, the promoter may direct expression of the polynucleotides encoding the replication-specific polypeptides in a specific tissue or may be otherwise under more precise environmental or developmental control. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Examples of plant promoters under developmental control include promoters that initiate transcription only (or primarily only) in certain tissues, such as fruit, seeds, or flowers. Promoters that direct expression of nucleic acids in flowers or seeds are particularly useful in the present invention. Suitable seed specific promoters include those derived from the following genes: MAC1 from maize (Sheridan et al. Genetics 142:1009-1020 (1996), Cat3 from maize (GenBank No. L05934, Abler et al. Plant Mol. Biol. 22:10131-1038 (1993), the gene encoding oleosin 18kD from maize (GenBank No. J05212, Lee et al. Plant Mol Biol. 26:1981-1987 (1994)), vivparous-1 from Arabidopsis (Genbank No. U93215), the gene encoding oleosin from Arabidopsis (Genbank No. Z17657), Atmycl from Arabidopsis (Urao et al. Plant Mol. Biol. 32:571-576 (1996), the 2s seed storage protein gene family from Arabidopsis (Conceicao et al. Plant 5:493-505 (1994)) the gene encoding oleosin 20kD from Brassica napus (GenBank No. M63985), napA from Brassica napus (GenBank No. J02798, Josefsson et al. JBL 26:12196-1301 (1987), the napin gene family from Brassica napus (Sjodahl et al. Planta 197:264-271 (1995), the gene encoding the 2S storage protein from Brassica napus (Dasgupta et al. Gene 133:301-302 (1993)), the genes encoding oleosin A (Genbank No. U09118) and oleosin B (Genbank No. U09119) from soybean and the gene encoding low molecular weight sulphur rich protein from soybean (Choi et al. Mol Gen, Genet. 246:266-268 (1995)).

A variety of promoters specifically active in vegetative tissues, such as leaves, stems, roots and tubers, can also be used to express the nucleic acids of the invention. For example, promoters controlling patatin, the major storage protein of the potato tuber, can be used, see, e.g., Kim (1994) Plant Mol. Biol. 26:603-615; Martin (1997) Plant J. 11:53-62. The ORF13 promoter from Agrobacterium rhizogenes which exhibits high activity in roots can also be used (Hansen (1997) Mol. Gen. Genet. 254:337-343. Other useful vegetative tissue-specific promoters include: the tarin promoter of the gene encoding a globulin from a major taro (Colocasia esculenta L. Schott) corm protein family, tarin (Bezerra (1995) Plant Mol. Biol. 28:137-144); the curculin promoter active during taro corm development (de Castro (1992) Plant Cell 4:1549-1559) and the promoter for the tobacco root-specific gene TobRB7, whose expression is localized to root meristem and immature central cylinder regions (Yamamoto (1991) Plant Cell 3:371-382).

Leaf-specific promoters, such as the ribulose biphosphate carboxylase (RBCS) promoters can be used. For example, the tomato RBCS1, RBCS2 and RBCS3A genes are expressed in leaves and light-grown seedlings, only RBCS1 and RBCS2 are expressed in developing tomato fruits (Meier (1997) FEBS Lett. 415:91-95). A ribulose bisphosphate carboxylase promoters expressed almost exclusively in mesophyll cells in leaf blades and leaf sheaths at high levels, described by Matsuoka (1994) Plant J. 6:311-319, can be used. Another leaf-specific promoter is the light harvesting chlorophyll a/b binding protein gene promoter, see, e.g., Shiina (1997) Plant Physiol. 115:477-483; Casal (1998) Plant Physiol. 116:1533-1538. The Arabidopsis thaliana myb-related gene promoter (Atmyb5) described by Li (1996) FEBS Lett. 379:117-121, is leaf-specific. The Atmyb5 promoter is expressed in developing leaf trichomes, stipules, and epidermal cells on the margins of young rosette and cauline leaves, and in immature seeds. Atmyb5 mRNA appears between fertilization and the 16 cell stage of embryo development and persists beyond the heart stage. A leaf promoter identified in maize by Busk (1997) Plant J. 11:1285-1295, can also be used.

Another class of useful vegetative tissue-specific promoters are meristematic (root tip and shoot apex) promoters. For example, the “SHOOTMERISTEMLESS” and “SCARECROW” promoters, which are active in the developing shoot or root apical meristems, described by Di Laurenzio (1996) Cell 86:423-433; and, Long (1996) Nature 379:66-69; can be used. Another useful promoter is that which controls the expression of 1-hydroxy-3-methylglutaryl coenzyme A reductase HMG2 gene, whose expression is restricted to meristematic and floral (secretory zone of the stigma, mature pollen grains, gynoecium vascular tissue, and fertilized ovules) tissues (see, e.g., Enjuto (1995) Plant Cell. 7:517-527). Also useful are kn1-related genes from maize and other species which show meristem-specific expression, see, e.g., Granger (1996) Plant Mol. Biol. 31:373-378; Kerstetter (1994) Plant Cell 6:1877-1887; Hake (1995) Philos. Trans. R. Soc. Lond. B. Biol. Sci. 350:45-51. For example, the Arabidopsis thaliana KNAT1 promoter. In the shoot apex, KNAT1 transcript is localized primarily to the shoot apical meristem; the expression of KNAT1 in the shoot meristem decreases during the floral transition and is restricted to the cortex of the inflorescence stem (see, e.g., Lincoln (1994) Plant Cell 6:1859-1876).

Examples of tissue-specific promoters for animal cells include the promoter for creatine kinase, which has been used to direct the expression of dystrophin cDNA expression in muscle and cardiac tissue (Cox, et al. Nature 364:725-729 (1993)) and immunoglobulin heavy or light chain promoters for the expression of suicide genes in B cells (Maxwell, et al. Cancer Res. 51:4299-4304 (1991)). An endothelial cell-specific regulatory region has also been characterized (Jahroudi, et al. Mol. Cell. Biol. 14:999-1008 (1994)). Amphotrophic retroviral vectors have been constructed carrying a herpes simplex virus thymidine kinase gene under the control of either the albumin or alpha-fetoprotein promoters (Huber, et al. Proc. Natl. Acad. Sci. U.S.A. 88:8039-8043 (1991)) to target cells of liver lineage and hepatoma cells, respectively.

The human smooth muscle-specific alpha-actin promoter is discussed in Reddy, et al., J. Cell Biology 265:1683-1687 (1990) which discloses the isolation and nucleotide sequence of this promoter, while Nakano, et al., Gene 99:285-289 (1991) discloses transcriptional regulatory elements in the 5′ upstream and the first intron regions of the human smooth muscle (aortic type) alpha-actin gene. Petropoulos, et al., J. Virol. 66:3391-3397 (1992)) disclose a comparison of expression of bacterial chloramphenicol transferase (CAT) operatively linked to either the chicken skeletal muscle alpha actin promoter or the cytoplasmic beta-actin promoter.

Exemplary tissue-specific expression elements for the liver include but are not limited to HMG-COA reductase promoter (Luskey, Mol. Cell. Biol. 7(5):1881-1893 (1987)); sterol regulatory element 1 (SRE-1; Smith et al. J. Biol. Chem. 265(4):2306-2310 (1990); phosphoenol pyruvate carboxy kinase (PEPCK) promoter (Eisenberger et al. Mol. Cell Biol. 12(3):1396-1403 (1992)); human C-reactive protein (CRP) promoter (Li et al. J. Biol. Chem. 265(7):4136-4142 (1990)); human glucokinase promoter (Tanizawa et al. Mol. Endocrinology 6(7):1070-81 (1992); cholesterol 7-alpha hydroylase (CYP-7) promoter (Lee et al. J. Biol. Chem. 269(20):14681-9 (1994)); beta-galactosidase alpha-2,6 sialyltransferase promoter (Svensson et al. J. Biol. Chem. 265(34):20863-8 (1990); insulin-like growth factor binding protein (IGFBP-1) promoter (Babajko et al. Biochem Biophys. Res. Comm. 196 (1):480-6 (1993)); aldolase B promoter (Bingle et al. Biochem J. 294(Pt2):473-9 (1993)); human transferrin promoter (Mendelzon et al. Nucl. Acids Res. 18(19):5717-21 (1990); collagen type I promoter (Houglum et al. J. Clin. Invest. 94(2):808-14 (1994)).

Exemplary tissue-specific expression elements for the prostate include but are not limited to the prostatic acid phosphatase (PAP) promoter (Banas et al. Biochim. Biophys. Acta. 1217(2):188-94 (1994); prostatic secretory protein of 94 (PSP 94) promoter (Nolet et al. Biochim. Biophys. ACTA 1089(2):247-9 (1991)); prostate specific antigen complex promoter (Kasper et al. J. Steroid Biochem. Mol. Biol. 47 (1-6):127-35 (1993)); human glandular kallikrein gene promoter (hgt-1) (Lilja et al. World J. Urology 11(4):188-91 (1993).

Exemplary tissue-specific expression elements for gastric tissue include those discussed in Tamura et al. FEBS Letters 298: (2-3):137-41 (1992).

Exemplary tissue-specific expression elements for the pancreas include but are not limited to pancreatitis associated protein promoter (PAP) (Dusetti et al J. Biol. Chem. 268(19):14470-5 (1993)); elastase 1 transcriptional enhancer (Kruse et al. Genes and Development 7(5):774-86 (1993)); pancreas specific amylase and elastase enhancer promoter (Wu et al Mol. Cell. Biol. 11(9):4423-30 (1991); Keller et al. Genes & Dev. 4(8):1316-21 (1990)); pancreatic cholesterol esterase gene promoter (Fontaine et al. Biochemistry 30(28):7008-14 (1991)).

Exemplary tissue-specific expression elements for the endometrium include but are not limited to the uteroglobin promoter (Helftenbein et al. Annal. NY Acad. Sci. 622:69-79 (1991)).

Exemplary tissue-specific expression elements for adrenal cells include but are not limited to cholesterol side-chain cleavage (SCC) promoter (Rice et al. J. Biol. Chem. 265:11713-20 (1990).

Exemplary tissue-specific expression elements for the general nervous system include but are not limited to gamma-gamma enolase (neuron-specific enolase, NSE) promoter (Forss-Petter et al. Neuron 5(2):187-97 (1990)).

Exemplary tissue-specific expression elements for the brain include but are not limited to the neurofilament heavy chain (NF-H) promoter (Schwartz et al. J. BioL Chem. 269(18):13444-50 (1994)).

Exemplary tissue-specific expression elements for lymphocytes include but are not limited to the human CGL-1/granzyme B promoter (Hanson et al. J. Biol. Chem. 266 (36):24433-8 (1991)); the terminal deoxy transferase (TdT), lambda 5, VpreB, and Ick (lymphocyte specific tyrosine protein kinase p561ck) promoter (Lo et al. Mol. Cell. Biol. 11(10):5229-43 (1991)); the humans CD2 promoter and its 3′ transcriptional enhancer (Lake et al. EMBO J. 9(10):3129-36 (1990)), and the human NK and T cell specific activation (NKG5) promoter (Houchins et al. Immunogenetics 37(2):102-7 (1993)).

Exemplary tissue-specific expression elements for the colon include but are not limited to pp6oc-src tyrosine kinase promoter (Talamonti et al. J. Clin. Invest 91(1):53-60 (1993)); organ-specific neoantigens (OSNs), mw 40 kDa (p40) promoter (Ilantzis et al. Microbiol. Immunol. 37(2):119-28 (1993)); colon specific antigen-P promoter (Sharkey et al. Cancer 73(3 supp.) 864-77 (1994)).

Exemplary tissue-specific expression elements for breast cells include but are not limited to the human alpha-lactalbumin promoter (Thean et al. British J. Cancer. 61(5):773-5 (1990))

Other tissue-specific promoters include the phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter.

Other elements aiding specificity of expression in a tissue of interest can include secretion leader sequences, enhancers, nuclear localization signals, endosmolytic peptides, etc. Preferably, these elements are derived from the tissue of interest to aid specificity.

c. Inducible Promoters

Examples of environmental conditions that may effect transcription by inducible promoters include anaerobic conditions, elevated temperature, a particular chemical compound or the presence of light. Such promoters are referred to here as “inducible” promoters. For instance, inducible promoters include the glucocorticoid-inducible promoter described in McNellis et al., Plant J. 14(2):247-57 (1998). U.S. Pat. No. 5,877,018 describes metal responsive and glucocorticoid-responsive promoter elements. Other examples include promoters induced in response to infection or disease.

5. Replication Cassettes of the Invention

A replication cassette is a polynucleotide sequence that can be replicated by the replication-specific polypeptides. In general, the replication cassette comprises an origin of replication, thereby allowing for the initiation of DNA replication on the cassette.

a. Origins of Replication

Origins of replication from phage, bacterial and eukaryotic cells are well known. See, e.g., Darnell, et al., MOLECULAR CELL BIOLOGY (Scientific American Books, 1986), page 549. An origin of replication is any cis-acting polynucleotide sequence at which the replication system can initiate DNA replication. The choice of which origin to use in the compositions and methods of the invention will depend from which replication system is used. Preferably, the origin of replication are derived from the same organism from which the polypeptides of the replication system are derived.

For example, to exemplify the invention, the Examples describe a replication system derived from phage T7 DNA replication factors. Therefore, the exemplified replication cassette comprises a T7 promoter (Genbank Accession No. V01146), which comprises a T7 origin of replication. See, e.g., Fuller et al., Biol Chem 260(5):3185-96 (1985). The T7 phiOR promoter acts as an origin of replication that is recognized by the T7 replication system discussed above.

b. Replication Cassette Genes

In some embodiments of the invention, the replication cassette comprises a gene encoding a polypeptide of interest. For instance, genes conferring resistance to pathogens (for example, insects, fungi, bacteria and viruses), storage protein genes, herbicide resistance genes, and genes involved in biosynthetic pathways can be inserted into the replication cassette. In some embodiments, expression signal sequences (for example, promoter and terminator regions) operably linked to the coding regions are also included in the replication cassettes of the invention. Promoters linked to the coding regions can be the same or a different promoter than the promoter linked to the polynucleotides of the replication system.

C. Sequence-Specific Recombination Site

In a preferred embodiment, the replication cassette comprises a polynucleotide sequence that is recognized by a sequence-specific recombinase. As discussed above, linear concatamers can be produced by replication of the replication cassettes. The recombination sequence is then processed by a recombinase, if present, thereby cleaving the concatamers. In some embodiments, after processing by the recombinase, the linear polynucleotides are converted into individual circular cassettes by the recombinase.

Recombination sequences typically have an orientation. In other words, they are not palindromes. The orientation of the recombination sites in relation to each other determines what recombination event takes place. The recombination sites may be in two different orientations: parallel (same direction) or opposite. When the recombination sites are in the opposite orientation with respect to each other, then the recombination event catalyzed by the recombinase is an inversion. When the recombination sites are in the parallel orientation, then any intervening sequence is excised, leaving a single recombination site. The remaining recombination site may or may not be altered, depending on the recombination fidelity of the recombinase.

One recombination system is the Cre-lox system. In the Cre-lox system, the recombination sites are referred to as “lox sites” and the recombinase is referred to as “Cre”. When lox sites are in parallel orientation (i.e., in the same direction), then Cre catalyzes a deletion of the intervening polynucleotide sequence. When lox sites are in the opposite orientation, the Cre recombinase catalyzes an inversion of the intervening polynucleotide sequence. Therefore, in some preferred embodiments, tandem repeats of parallel lox sites are provided in the replication cassette. In the presence of the Cre recombinase, the linear concatamers of the replicated replication cassette are deconcatamerized and rendered into individual circles. This system works in various host cells, including Saccharomyces cerevisiae (Sauer, B., Mol Cell Biol. 7:2087-2096 (1987)); mammalian cells (Sauer, B. et al., Proc. Natl Acad. Sci. USA 85:5166-5170 (1988); Sauer, B. et al., Nucleic Acids Res. 17:147-161 (1989)); and plants such as tobacco (Dale, E. et al., Gene 91:79-85 (1990)) and Arabdiopsis (Osborne, B. et al., Plant J. 7(4):687-701 (1995)). Use of the Cre-lox recombinase system in plants is also described in U.S. Pat. No. 5,527,695 and PCT application No. WO 93/01283. Several different lox sites are known, including lox511 (Hoess R. et al., Nucleic Acids Res. 14:2287-2300 (1986)), lox66, lox71, lox76, lox75, lox43, lox44 (Albert H. et al., Plant J. 7(4): 649-659 (1995)).

Several other recombination systems are also suitable for use in the invention. These include, for example, the FLP/FRT system of yeast (Lyznik, L. A. et al., Nucleic Acids Res. 24(19):3784-9 (1996)), the Gin recombinase of phage Mu (Crisona, N.J. et al., J. Mol. Biol. 243(3):437-57 (1994)), the Pin recombinase of E. coli (see, e.g., Kutsukake K, et. aL, Gene 34(2-3):343-50 (1985)), the PinB, PinD and PinF from Shigella (Tominaga A et al., J. Bacteriol. 173(13):4079-87 (1991)), and the R/RS system of the pSR1 plasmid (Araki, H. et al., J. Mol. Biol. 225(1):25-37 (1992)). Thus, recombinase systems are available from a large and increasing number of sources. Recombinase systems may be employed in the cells of any organism that can be transformed with nucleic acids.

6. Isolation of Thepolynucleotides of the Invention

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989) or Ausubel et al., eds., Current Protocols, a joint venture between Greene Publishing Associates, Inc. and John Wiley & Sons, Inc., (1995 Supplement) (Ausubel).

The isolation of nucleic acids of the invention may be accomplished by a number of techniques. For instance, oligonucleotide probes based on the sequences disclosed here can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ, such as ovules, and a cDNA library which contains a gene transcript of the invention is prepared from the MRNA. Alternatively, cDNA may be prepared from mRNA extracted from other tissues in which genes of the invention or homologs are expressed.

The cDNA or genomic library can then be screened using a probe based upon the sequence of a cloned gene of the invention. Probes may be used to hybridize with genomic DNA or cDNA sequences to isolate homologous genes in the same or different plant species. Alternatively, antibodies raised against an polypeptide of the invention can be used to screen an mRNA expression library.

Alternatively, the nucleic acids of interest can be amplified from nucleic acid samples using amplification techniques. For instance, polymerase chain reaction (PCR) technology can be used to amplify the sequences of the genes of the invention directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired MRNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990).

Appropriate primers and probes for identifying sequences of the invention from plant tissues are generated from comparisons of the sequences provided here with other related genes. Using these techniques, one of skill can identify conserved regions in the nucleic acids of the invention to prepare the appropriate primer and probe sequences. Primers that specifically hybridize to conserved regions in genes of the invention can be used to amplify sequences from widely divergent plant species.

The amplification conditions are typically as follows. Reaction components: 10 mM Tris-HCl, pH 8.3, 50 mM potassium chloride, 1.5 mM magnesium chloride, 0.001% gelatin, 200 μM dATP, 200 μM dCTP, 200 μM dGTP, 200 μM dTTP, 0.4 μM primers, and 100 units per ml Taq polymerase. Program: 96 C. for 3 min., 30 cycles of 96 C. for 45 sec., 50 C. for 60 sec., 72 for 60 sec, followed by 72 C. for 5 min.

Standard nucleic acid hybridization techniques using the conditions disclosed above can then be used to identify full-length cDNA or genomic clones.

7. Transformation

a. Transformation of Plants

Recombinant DNA vectors suitable for transformation of cells are well known in the art and can be prepared to introduce constructs if the invention into various organsims. Techniques for transforming a wide variety of flowering plant species are well known and described in the technical and scientific literature. See, for example, Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens—mediated transformation techniques, including disarming and use of binary vectors, are well described in the scientific literature. See, for example, Horsch et al. Science 233:496-498 (1984), and Fraley et al. Proc. Natl. Acad. Sci. USA 80:4803 (1983).

Transformed plant cells which are derived by any of the above transformation techniques can be propagated as cell cultures or can be cultured to regenerate a whole plant which possesses the transformed genotype and thus the desired phenotype such as increased seed mass. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

The nucleic acids of the invention can be used to confer desired traits on essentially any plant. Thus, the invention has use over a broad range of plants, including species from the genera Anacardium, Arachis, Asparagus, Atropa, Avena, Brassica, Citrus, Citrullus, Capsicum, Carthamus, Cocos, Coffea, Cucumis, Cucurbita, Daucus, Elaeis, Fragaria, Glycine, Gossypium, Helianthus, Heterocallis, Hordeum, Hyoscyamus, Lactuca, Linum, Lolium, Lupinus, Lycopersicon, Malus, Manihot, Majorana, Medicago, Nicotiana, Olea, Oryza, Panieum, Pannesetum, Persea, Phaseolus, Pistachia, Pisum, Pyrus, Prunus, Raphanus, Ricinus, Secale, Senecio, Sinapis, Solanum, Sorghum, Theobromus, Trigonella, Triticum, Vicia, Vitis, Vigna, and Zea.

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Seed obtained from plants of the present invention can be analyzed according to well known procedures to identify plants with the desired trait. If antisense or other techniques are used to control gene expression, Northern blot analysis can be used to screen for desired plants.

b. Transfromation of Animal Cells and Production of Transgenic Animals

Transformation of Cells

Standard transfection methods are used to produce bacterial, mammalian, yeast or insect cell lines that express large quantities of a protein of interest, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132:349-351 (1977); Clark-Curtiss & Curtiss,i Methods in Enzymology 101:347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Sambrook et al., Molecular Cloning, A Laboratory Manual (2nd ed. 1989)).

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the CMV promoter, SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase and dihydrofolate reductase. Alternatively, high yield expression systems not involving gene amplification are also suitable, such as using a baculovirus vector in insect cells, with a protein-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are preferably chosen such that they do not interfere with the replication of the DNA in eukaryotic cells, if necessary.

Production of Transgenic Animals

The methods and compositions of the present invention can also be applied to transgenic animals. One method of introducing a vector into the animal's germ line involves using embryonic stem (ES) cells as recipients of the expression vector. ES cells are pluripotent cells directly derived from the inner cell mass of blastocysts (Evans et al., Nature 292:154-156 (1981); Martin Proc. Natl. Acad Sci. USA 78:7634-7638 (1981); Magnuson et al., J. Embryo. Exp. Morph. 81:211-217 (1982); Doetzchman et al., Dev. Biol., 127:224-227 (1988)), from inner cell masses (Tokunaga et al., Jpn. J. Anim. Reprod., 35:113-178 (1989)), from disaggregated morulae (Eistetter, Dev. Gro. Differ., 31:275-282 (1989)) or from primordial germ cells (Matsui et al., Cell 70:841-847 (1992); and Resnick et al., Nature 359:550-551 (1992)). Vectors can be introduced into ES cells using any method which is suitable for gene transfer into cells, e.g., by transfection, cell fusion, electroporation, microinjection, DNA viruses, and RNA viruses (Johnson et al., Fetal Ther., 4 (Suppl. 1):28-39 (1989)). Once the expression vector has been introduced into an ES cell, the modified ES cell is then introduced back into the embryonic environment for expression and subsequent transmission to progeny animals. The most commonly used method is the injection of several ES cells into the blastocoel cavity of intact blastocysts (Bradley et al., Nature 309:225-256 (1984)). Alternatively, a clump of ES cells may be sandwiched between two eight-cell embryos (Bradley et al., in TERATOCARCINOMAS AND EMBRYONIC STEM CELLS: A PRACTICAL APPROACH, Robertson E. J. (ed.), IRL Press, Oxford, U.K. (1987), pp. 113-151; and Nagy et al., Development 110:815-821 (1990)). Both methods result in germ line transmission at high frequency.

Transgenes may also be introduced into ES cells by retrovirus-mediated transduction or by micro-injection. Transfected ES cells which contain the transgene may be subjected to various selection protocols to enrich for ES cells which have integrated the transgene assuming that the transgene provides a means for such selection. Alternatively, the polymerase chain reaction may be used to screen for ES cells which have integrated the transgene. This technique obviates the need for growth of the transfected ES cells under appropriate selective conditions prior to transfer into the blastocoel.

Transfected ES cells can thereafter colonize an embryo following their introduction into the blastocoel of a blastocyst-stage embryo and contribute to the germ line of the resulting chimeric animal. For review see Jaenisch, Science 240:1468-1474 (1988).

Alternatively, targeting vectors or transgenes may be microinjected into oocytes to generate transgenic animals. Once the expression vector has been injected into the fertilized egg cell, the cell is implanted into the uterus of a pseudopregnant female and allowed to develop into an animal. Heterozygous and homozygous animals can then be produced by interbreeding founder transgenics. This method has been successful in producing transgenic mice, sheep, pigs, rabbits and cattle (See, Jaenisch, supra; Hammer et al., J. Animal Sci., 63:269 (1986); Hammer et al., Nature 315:680-683 (1995); and Wagner et al., Theriogenology 21:29 (1984)).

Alternative methods for the production include the infection of embryos with retroviruses or with retroviral vectors. Infection of both pre- and post-implantation mouse embryos with either wild-type or recombinant retroviruses has been reported Jaenisch, Proc. Natl. Acad. Sci. USA 73:1260-1264 (1976); Jaenisch et al. Cell 24:519 (1981); Stuhlmann et al. Proc. Natl. Acad. Sci. USA 81:7151 (1984); Jahner et al. Proc. Natl. Acad Sci. USA 82:6927-6931 (1985); Van der Putten, et al. Proc. Natl. Acad Sci. USA 82:6148-6152 (1985); Stewart, et al. (1987) EMBO J. 6:383-388. The resulting transgenic animals are typically mosaic for the transgene since incorporation occurs only in a subset of cells which form the transgenic animal.

An alternative means for infecting embryos with retroviruses is the injection of virus or virus-producing cells into the blastocoele of mouse embryos Jahner, D. et al. Nature 298:623-628 (1982). As is the case for infection of eight cell stage embryos, most of the founders produced by injection into the blastocoele will be mosaic. The introduction of transgenes into the germline of mice has been reported using intrauterine retroviral infection of the midgestation mouse embryo Jahner, D. et al., supra. This technique suffers from a low efficiency of generation of transgenic animals and in addition produces animals which are mosaic for the transgene.

Other methods of generating transgenic animals are discussed in U.S. Pat. Nos. 6,080,912 and 5,945,577 and Great Britain patents GB2331751 and GB2318578.

7. Selection Of Organisms Without The Replication Cassette

Genetic mating can be designed to result in organisms without the transgene or replication cassettes. For example, back-crossing of the transgenic organism to a wild type organism will lead segregation of the transgene according to Mendelian genetics. Thus, after the recombination cassette of the invention has been introduced into an organism by homologous recombination, the resulting organism can be crossed to wild type organisms to select for organisms that lack the original transgene but include the DNA introduced by homologous recombination.

In one embodiment, a transgene of interest (e.g., a selectable marker gene) is deleted from an organism, e.g., a plant, by homologous recombination. Progeny of the organism can subsequently be selected that lack both the transgene and the replication cassette. Those of skill in the art will recognize that by mating the organism to an second organism without the replication cassette, progeny with the desired genotype can be selected.

8. Measurement of DNA Replication

Replication of specific DNA constructs (e.g., the replication cassette) can be measured in a number of ways known to those of skill in the art. Diagnostic amplification of a particular polynucleotide using the polymerase chain reaction is useful to determine whether replication has occurred. For example, amplification primers can be designed to amplify only those target fragments that have been replicated and subsequently circularized by the recombinase (e.g., by designing primers that amplify “out” from the linear replication cassette).

Alternatively, hybridization experiments can be performed to quantify the DNA in a sample. Quantification of hybridization of a probe directed to the replication cassette compared to hybridization of a second probe to a control sequence within the DNA sample will provide an estimate of the quantity of replication cassettes in the sample relative to background chromosomal copy number.

### B. Selected Applications of the Methods and Compositions of the Invention

Those of skill in the art will recognize that initiating replication and generating increased DNA copies of replication cassettes of the invention is useful in many different applications. The following list of applications is not intended to limit the invention, but is merely to exemplify various embodiments.

1. Homologous Recombination

Homologous recombination can occur in a cell when two DNA molecules are recombined at sites where their DNA is similar. See Alberts et al., supra; Mengiste, T., et al., Biol Chem 380(7-8): 749-58 (1999). Mechanisms of homologous recombination in plants has been described, e.g., in Puchta, et al. Proc. Natl. Acad. Sci. USA 93(10):5055-60 (1996). One advantage of the present invention is that efficiency of homologous recombination reactions is improved with higher copy number of similar DNA sequences. Therefore, by increasing the copy number of DNA that is similar to chromosomal DNA of the host cell (i.e., comprised by the replication cassette), it is possible to increase the efficiency of homologous recombination.

For best results, the copy number of the replication cassette is increased when homologous recombination occurs, i.e., during meiosis. Therefore, replication of the cassette is preferably induced during meiosis (e.g., via induction of meiosis-specific promoters expressing the polynucleotides of the replication system). Alternatively, constitutive promoters may be used to induce expression in all or nearly all tissues of an organism.

To introduce the nucleic acid constructs of the invention, recombinant DNA vectors suitable for transformation of cells are prepared. Techniques for transforming a wide variety of higher plant species are well known and described in the technical and scientific literature. See, for example, Weising et al. Ann. Rev. Genet. 22:421-477 (1988). A DNA sequence of interest fused to a reporter sequence, will be prepared according well known techniques and incorporated into an appropriate vector that lacks transcriptional and translational initiation regulatory sequences, such transcription of the fusion sequence will occur only in the event of homologous recombination.

Well known recombinant DNA methods are used to construct recombinant DNA molecules that comprise a targeting DNA containing sequences at least substantially identical to the target plant nucleotide sequence. Thus, the sequences used in the vectors of the invention will depend in large part on the target nucleotide sequence. The targeting DNA will typically be a variant of the target gene so that homologous recombination results in replacement of the target gene (or a portion of the gene) with the variant form. Incorporation of the targeting sequence by homologous recombination will result in expression of the reporter sequence, thereby allowing detection of the homologous recombination event (e.g., Jelesko, J., et al., P.N.A.S. USA, 96:10302-10307 (1999) and PCT WO 00/09728).

The particular endogenous sequence targeted in the methods of the invention is not a critical aspect of the invention. Examples of genes that can be targeted using the present invention include genes conferring resistance to pathogens (for example, insects, fungi, bacteria and viruses), storage protein genes, herbicide resistance genes, and genes involved in biosynthetic pathways. Any part of the target gene can be modified. Thus, expression signal sequences (for example, promoter and terminator regions) and transcribed regions that encode a specific polypeptide can be targeted using the constructs of the invention.

2. Modulation of Gene Expression

In another embodiment, the invention provides for temporal and spatial regulation of gene expression and gene silencing. Expression can be controlled on at least two levels. First, a promoter operably linked to a gene of interest can be chosen such that the expression of the gene is expressed in a preferred temporal or spatial pattern. Second, the DNA copy number of the replication cassette is controlled with the appropriate promoters operably linked to the polynucleotides of the replication system, thereby increasing replication of the replication cassette.

The compositions and method disclosed here can be used to design nucleic acids useful to inhibit expression of genes in organisms, including plants. For instance, antisense technology can be conveniently used. To accomplish this, a nucleic acid segment from the desired gene is cloned and operably linked to a promoter so that the antisense strand of RNA will be transcribed. The construct is then transformed into plants and the antisense strand of RNA is produced. In plant cells, it has been suggested that antisense suppression can act at all levels of gene regulation including suppression of RNA translation (see, Bourque Plant Sci. (Limerick) 105: 125-149 (1995); Pantopoulos In Progress in Nucleic Acid Research and Molecular Biology, Vol. 48. Cohn, W. E. and K. Moldave (Ed.). Academic Press, Inc.: San Diego, Calif., USA; London, England, UK. p. 181-238; Heiser et al. Plant Sci. (Shannon) 127: 61-69 (1997)) and by preventing the accumulation of mRNA which encodes the protein of interest, (see, Baulcombe Plant Mol. Bio. 32:79-88 (1996); Prins and Goldbach Arch. Virol. 141: 2259-2276 (1996); Metzlaff et al. Cell 88: 845-854 (1997), Sheehy et al., Proc. Nat. Acad. Sci. USA, 85:8805-8809 (1988), and Hiatt et al., U.S. Pat. No. 4,801,340).

The nucleic acid segment to be introduced generally will be substantially identical to at least a portion of the endogenous gene or genes to be repressed. The sequence, however, need not be perfectly identical to inhibit expression. The vectors of the present invention can be designed such that the inhibitory effect applies to other genes within a family of genes exhibiting identity or substantial identity to the target gene.

For antisense suppression, the introduced sequence also need not be full length relative to either the primary transcription product or fully processed mRNA. Generally, higher identity can be used to compensate for the use of a shorter sequence. Furthermore, the introduced sequence need not have the same intron or exon pattern, and identity of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of about 500 to about 3500 nucleotides is especially preferred.

A number of gene regions can be targeted to suppress gene expression. The targets can include, for instance, the coding regions, introns, sequences from exon/intron junctions, 5′ or 3′ untranslated regions, and the like.

Another well-known method of suppression is sense co-suppression. Introduction of nucleic acid configured in the sense orientation has been recently shown to be an effective means by which to block the transcription of target genes. For an example of the use of this method to modulate expression of endogenous genes (see, Assaad et al. Plant Mol. Bio. 22: 1067-1085 (1993); Flavell Proc. Natl. Acad. Sci. USA 91: 3490-3496 (1994); Stam el al. Annals Bot. 79: 3-12 (1997); Napoli et al., The Plant Cell 2:279-289 (1990); and U.S. Pat. Nos. 5,034,323, 5,231,020, and 5,283,184).

The suppressive effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting identity or substantial identity.

For co-suppression, the introduced sequence, needing less than absolute identity, also need not be full length, relative to either the primary transcription product or fully processed mRNA. This may be preferred to avoid concurrent production of some plants that over-express the introduced sequence. A higher identity in a sequence shorter than full-length compensates for a longer, less identical sequence. Furthermore, the introduced sequence need not have the same intron or exon pattern, and identity of non-coding segments will be equally effective. Normally, a sequence of the size ranges noted above for antisense regulation is used. In addition, the same gene regions noted for antisense regulation can be targeted using co-suppression technologies.

In some embodiments, genes corresponding to plant virus genes (coat protein, replicase, etc.) can be expressed in plants, thereby disrupting viral gene expression and conferring resistance to one or more virus types. See, e.g., Smith et al., Plant Cell 6(10):1441-53 (1994). In some embodiments, a pathogen or virus-inducible promoter is operably linked to the viral gene in the replication cassette, thereby limiting expression of the gene to occurrences of viral infections.

3. Enhanced Transformation Frequency

DNA repair mechanisms have been implicated in transformation of plants in general, and, in Agrobacterium-mediated transformation in particular. See, e.g., Sonti et al., Proc Natl Acad Sci USA 92(25):11786-90 (1995) and Reiss, B. et al., Proc Natl Acad Sci USA 97(7):3358-63 (2000). The methods and composition of the invention are useful for stimulating DNA repair mechanisms of plant cells, thereby improving the efficiency of transformation of plants.

DNA repair mechanisms are initiated by single and double-stranded DNA breaks. See, e.g., Lao Y, et al. Biochemistry 39(5):850-9 (2000). Therefore, by inducing replication of the replication cassette, numerous DNA strands can be replicated in the nucleus, thereby stimulating DNA repair mechanisms and resulting in plant cells more likely to integrate introduced transgenes into their chromosomes. To produce the most single and double-stranded DNA ends, and thereby provide optimum stimulation of a cell's DNA repair mechanisms, replication cassettes without recombination sites are preferred. In these embodiments, linear, rather than circular, copies of the replication cassette are produced, thereby creating numerous DNA ends. By controlling the timing of the replication of the replication cassettes, it is possible to induce replication, and thus DNA repair mechanisms, when plant cells are most likely to be transformed or be susceptible to transformation.

### EXAMPLES

The following examples are offered to illustrate, but not to limit the claimed invention.

### Example 1

Cloning of Bacteriophage DNA Replication Genes

This example demonstrates the cloning of DNA replication genes into constructs useful for use of the present invention.

The polymerase chain reaction (PCR) is used to modify 5′ and 3′ ends of poynucleotides encoding T7 RNA polymerase, T7 gene 4 (helicase and primase), T7 DNA polymerase and TrxA (E. coli thioredoxin) to create modular cloning cassettes to simplify construction of several different constructs for different purposes. Similarly, the T7 2.5 gene (encoding a single-stranded DNA binding protein) is also amplified and cloned into an expression cassette. Each construct is created to have common restriction sites at their 5′ and 3′ ends. All ATG start codons are modified to be part of a NcoI site and the 3′ end of all T7 replication genes are modified to consist of a Bg/II-STOP-(XbaI or SpeI site compatible with NheI site in pCAMBIA vectors (CAMBIA, GPO Box 3200, Canberra, ACT 2601, Australia)). The Bg/II site precedes endogenous stop codons and leads to an in-frame fusion with GUS reporter gene in pCAMBIA vectors.

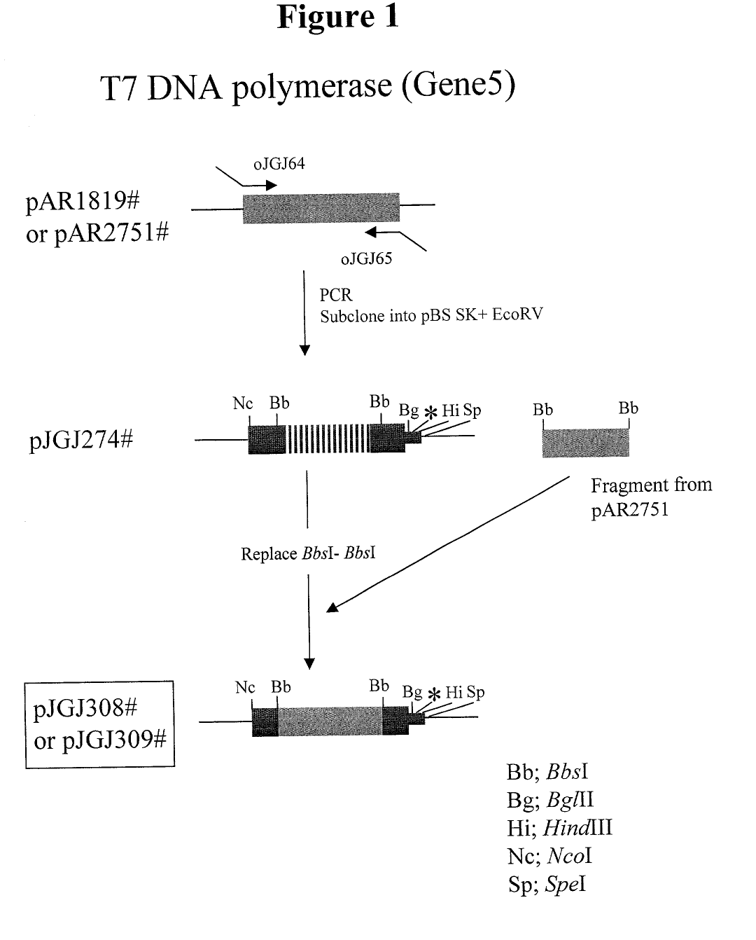

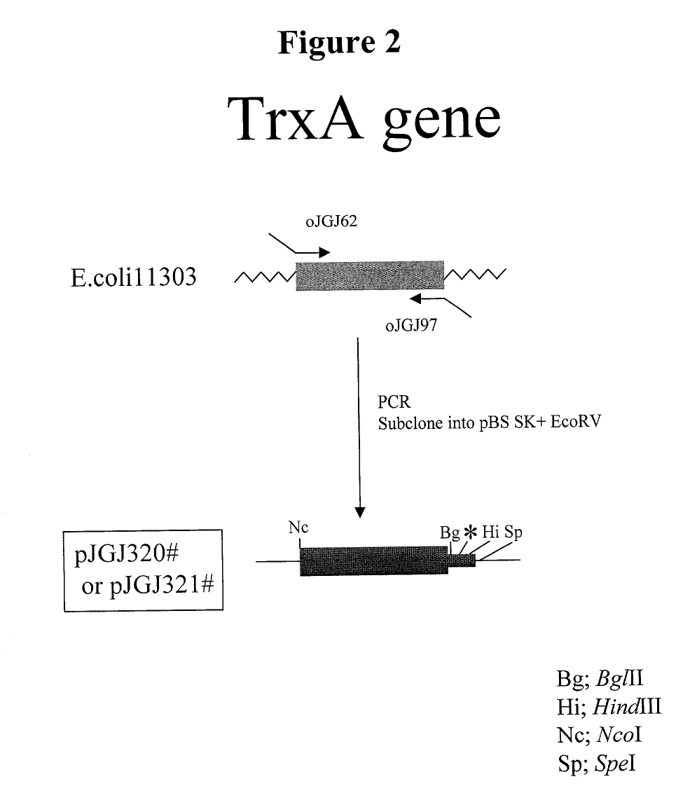

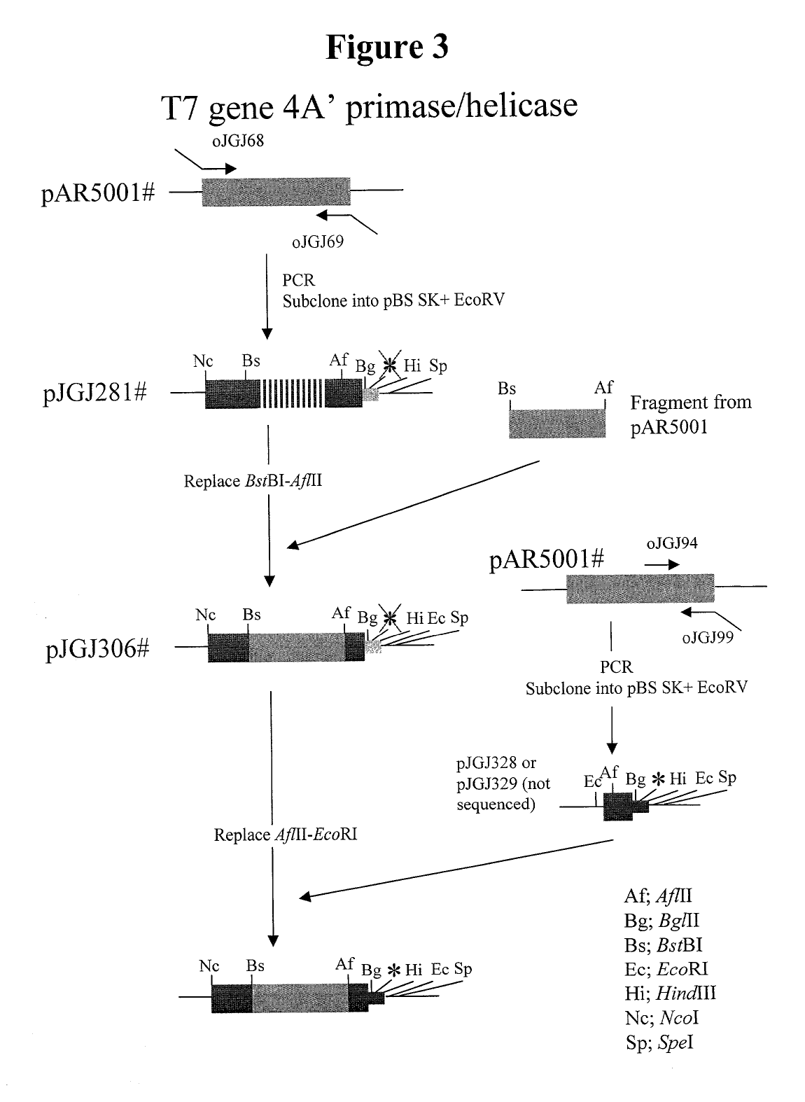

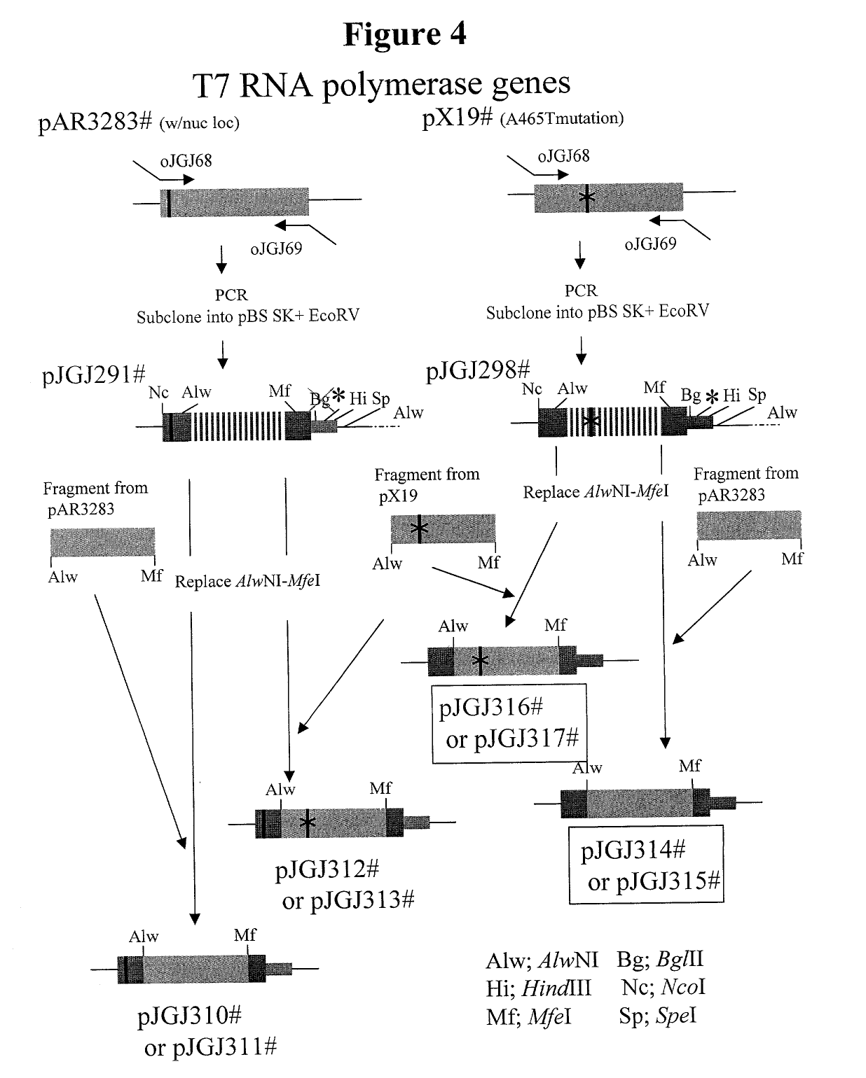

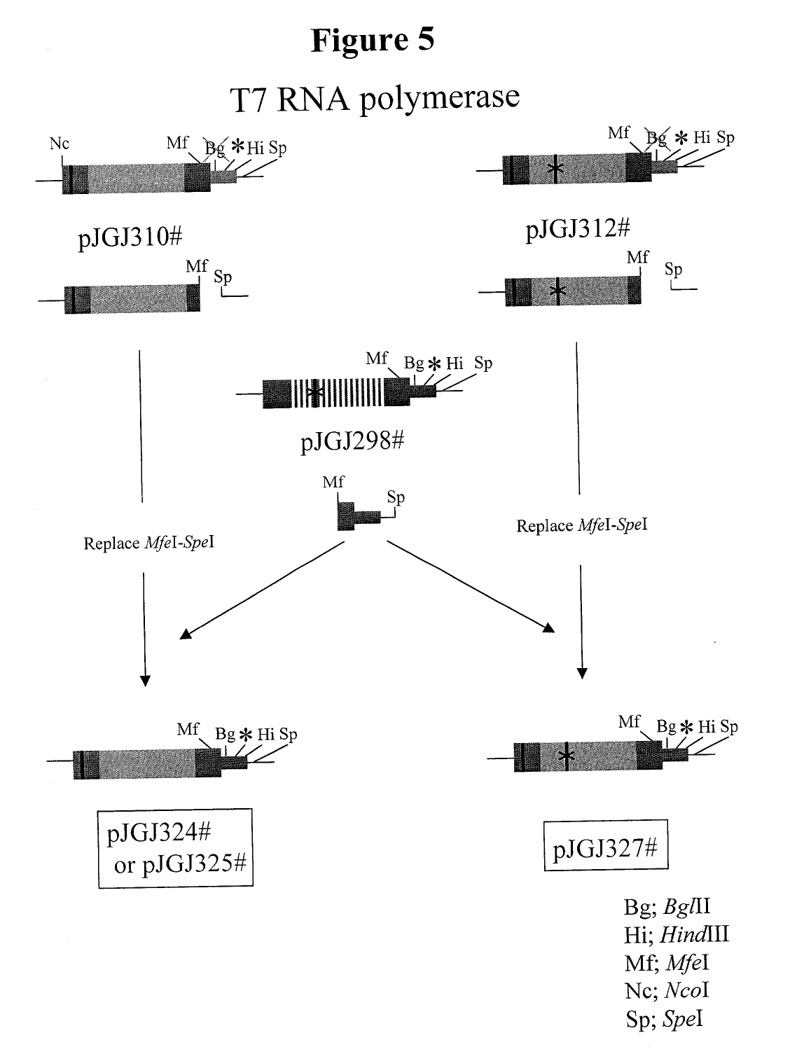

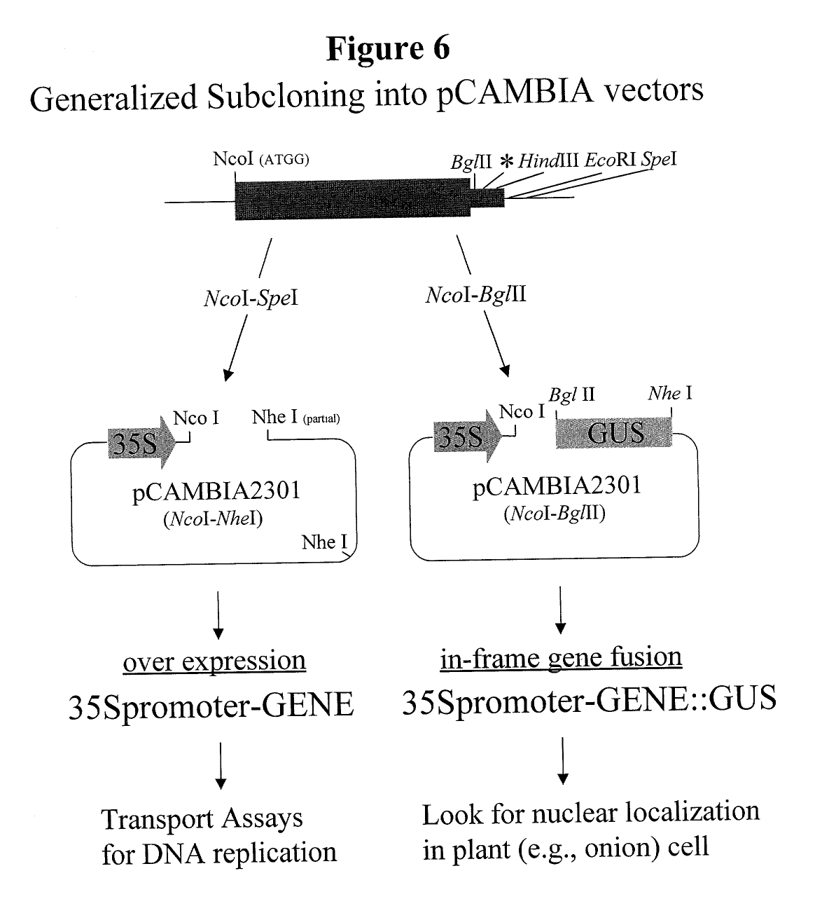

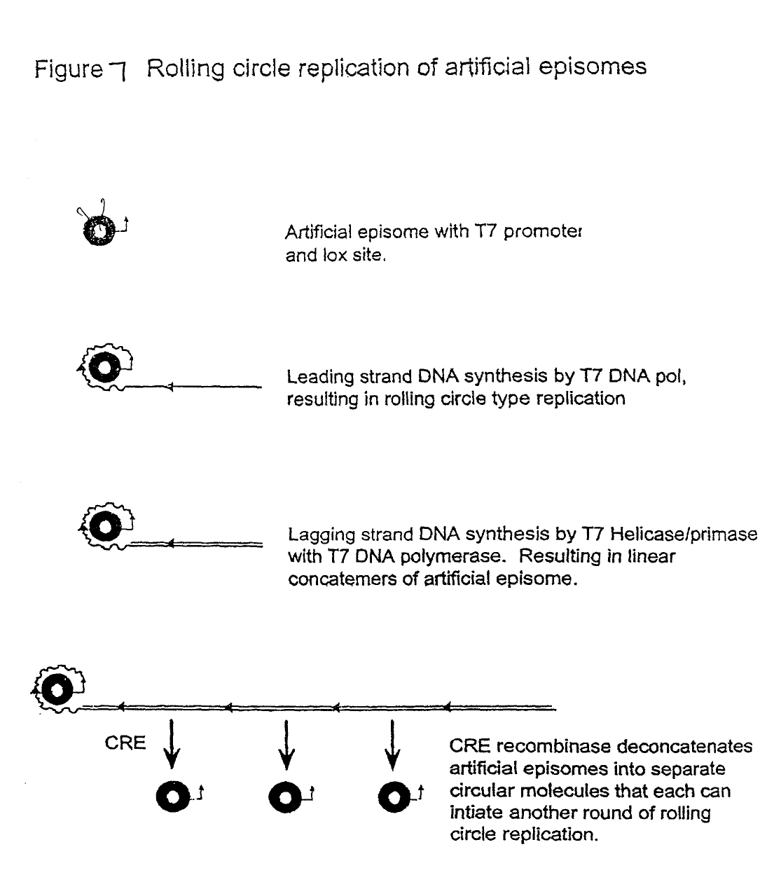

A cloning strategy for creating the replication genes is depicted in FIGS. 1-5. FIG. 6 depicts cloning of the replication genes into the pCAMBRIA2301 plasmid construct, thereby operably linking the CaMV 35S promoter to each construct.

### Example 2

Test of Artificial Plasmid/Replicon

This example demonstrates construction of a replication cassette.

A CaMV 35S promoter-Luciferase gene is cloned into pJGJ185 containing direct lox repeats flanking the multi-cloning site of the plasmid. A T7 promoter directing transcription towards the 5′ lox site is located between the CaMV 35S promoter and the 5′ lox site. This construct is used in transient gene expression studies to test for DNA replication as a circular plasmid/replicon in plants.

The construct is designed to show basal levels of the Luciferase reporter gene. However, if increased copies of this construct are produced, it will result in increased levels of Luciferase activity proportional to DNA concentration.

### Example 3

Direct Testing of in Planta T7 Mediated Replication of Artificial Plasmids

This example demonstrates that DNA replication is initiated by the constructs of the invention.

These experiments describe how one can amplify the copy number of the target sequence mediated by phage T7 replication machinery transiently expressed in plant cells. The T7 gene products will initiate a mRNA from the T7 promoter on the target site. This will serve as the initial primer for the T7 DNA polymerase: :TRXA complex that will perform leading strand DNA synthesis. The lagging strand DNA synthesis will be mediated by the Gene 4 product (helicase/primase) making short Okazaki fragments that will be extended by the T7 DNA polymerase::TRXA complex. A linear target sequence will not fully undergo DNA replication of sequences 3′ to T7 promoter; however, if the molecule is circularized by CRE-lox site specific recombination the replication complex should undergo a rolling circle mode of DNA replication and further deconcatenation by CRE-lox.

Tungsten particles are coated with a linear target DNA plasmid, CaMV35S prom-T7 RNA polymerase, CaMV35S prom-T7 gene 4, CaMV35S prom-T7 DNA polymerase, and GaMV35S prom-TrxA and bombarded into tobacco leaves by particle acceleration as described in, e.g., Millar, A. J., Plant Mol Biol Reporter, 10:324-337 (1992). Following bombardment, luciferase activity is measured.

Similarly, tungsten particles, which are further coated with a CaMV35S prom-CRE construct, are also bombarded into leaves. As described above, a significant increase in luciferase activity is expected to occur in the presence of CRE. An increase in LUC DNA content is confirmed using quantitative PCR.

### Example 4

Nuclear Localization of T7 Replication Gene Products

To improve nuclear localization of the T7 replication gene products, a 36 base pair nuclear localization signal sequence from SV40 T antigen (Dunn et al., Gene;68(2):259-66 (1988)) is fused to the amino terminus of the constructs. The replication genes are also fused to a GUS/green fluorescent protein (GFP) reporter gene fusion to monitor nuclear localization. The constructs used are: CaMV35S prom-T7 RNA polymerase::(BglII)::GUS-mGFP, CaMV35S prom-T7 gene 4::(BglII)::GUS-mGFP, CaMV35S prom-T7 DNA polymerase::(BglII)::GUS-m GFP, CaMV35S prom-TrxA::(BglII)::GUS-mGFP and CaMV35S prom-T7 gene 2.5::(BglII)::GUS-mGFP. “mGFP” is a modified form of GFP with several altered codons for optimum expression in plant cells. These constructs are bombarded into onion cells, which are then tested for GUS and GFP activity to determine the extent of nuclear localization.

### Example 4

Induction of Homologous Recombination

To demonstrate the improved efficiency of homologous recombination using the present invention, a candidate target gene, era1, is chosen (Cutler et al., Science 273(5279):1239-41 (1996)).

First, the following construct is created: lox-eral::LUC-3′ genomic DNA-T7 promoter-lox. Transgenic Arabidopsis thaliana plants carrying the constructs are developed and transgenic plants with single-copy insertions are identified.

Second, T7 replication genes (T7 RNA polymerase, T7 gene 4 (helicase and primase), T7 DNA polymerase, and TrxA) and Cre constructs are fused eithe the AtDMC1 (Klimyuk et al., Plant J. 11(1):1-14 (1997)) or AtSYN1 (Bai, X, et al., Plant Cell, 11:417-430 (1999)) promoters. The constructs are subsequently combined to create a multi-gene expression cassette. The construct is then transformed into plants and plants with single copies of the transgenes are identified.

The era1 plants are then mated to a plant comprising the T7 replication constructs and F2 plants are generated and screened for luciferase activity. Luciferase activity should only be present in F2 plants where the ERA1 gene and linked LUC gene has been exchanged into the plant's genome by homologous recombination.

True recombination events can be confirmed by selecting plants with LUC activity and then using molecular techniques to determine the structure of the ERA1 gene. For example, Long and Accurate PCR (LA-PCR) amplification of the endogenous er al gene can be compared with the ERA1 gene from the LUC positive plant. The LUC positive plant should have a larger product. The LA-PCR fragment can also be nucleotide sequenced to confirm and further characterize the homologous recombination event that has occurred. Southern hybridization can also be used to detect restriction fragment length polymorphisms (RFLP) between the wild type ERA1 gene and the candidate homologous recombinant.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of regulating the replication of a DNA molecule, the method comprising,
introducing into a eukaryotic cell,
a) a replication cassette comprising an origin of replication; and
b) a replication system comprising
i) a polynucleotide encoding a polypeptide with RNA polymerase activity;
ii) a polynucleotide encoding a polypeptide with DNA polymerase activity;
iii) a polynucleotide encoding a polypeptide with DNA belicase activity and;
iv) a polynucleotide encoding a polypeptide with DNA primase activity;
wherein the polynucleotide encoding each polypeptide is operably linked to a eukaryotic replication promoter, thereby initiating replication of the replication cassette independent from chromosomal DNA replication.

2. The method of claim 1, wherein the replication system comprises a polynucleotide encoding each of the following polypeptides: T7 RNA polymerase, T7 gene 4 protein, T7 DNA polymerase and TrxA.

3. The method of claim 2, wherein the eukaryotic cell is a plant cell.

4. The method of claim 2, wherein the eukaryotic cell is a mammalian cell.

5. The method of claim 2, wherein the origin of replication is a T7 bacteriophage origin of replication.

6. The method of claim 2, wherein the replication cassette comprises a T7 promoter.

7. The method of claim 2, wherein the replication cassette comprises an expression cassette.

8. The method of claim 2, wherein the expression cassette comprises a polynucleotide operably linked to an expression promoter in an antisense orientation.

9. The method of claim 2, wherein the expression cassette comprises a polynucleotide operably linked to an expression promoter in a sense orientation.

10. The method of claim 2, wherein the replication cassette comprises at least 200 base pairs of DNA that is at least 70% identical to chromosomal DNA in the eukaryotic cell.

11. The method of claim 10, wherein the replication cassette comprises at least 200 base pairs of DNA that is identical to chromosomal DNA in the eukaryotic cell.

12. The method of claim 2, wherein the replication cassette comprises a recombination sequence.

13. The method of claim 12, wherein the recombination sequence is a lox sequence.

14. The method of claim 2, wherein the replication system polynucleotide(s) further encode a sequence-specific recombinase operably linked to a promoter.

15. The method of claim 14, wherein the sequence-specific recombinase is the Cre recombinase.

16. The method of claim 2, wherein the eukaryotic replication promoter is tissue-specific.

17. The method of claim 2, wherein the eukaryotic replication promoter is constitutive.

18. The method of claim 2, wherein the eukaryotic replication promoter is meiosis-specific.

19. The method of claim 2, wherein the eukaryotic replication promoter is inducible.

20. The method of claim 2, wherein at least one of the replication system polynucleotide(s) encoding T7 RNA polymerase, T7 gene 4 protein, T7 DNA polymerase and TrxA encode a nuclear localization signal.

21. The method of claim 20, wherein all of the replication system polynucleotide(s) encoding T7 RNA polymerase, T7 gene 4 protein, T7 DNA polymerase and TrxA encode a nuclear localization signal.

22. The method of claim 2, wherein the number of copies of the replication cassette is increased.

23. A eukaryotic organism comprising a polynucleotide encoding each of the following polypeptides: T7 RNA polymerase, T7 gene 4 protein, T7 DNA polymerase and TrxA, wherein the polynucleotide encoding each polypeptide is operably linked to a eukaryotic replication promoter.

24. The eukaryotic organism of claim 23, wherein the organism is a plant.

25. The plant of claim 24, further comprising a replication cassette comprising a bacteriophage T7 origin of replication.

26. The plant of claim 24, wherein the replication cassette comprises an expression cassette.

27. The plant of claim 24, wherein the expression cassette comprises a polynucleotide operably linked to an expression promoter in an antisense orientation.

28. The plant of claim 24, wherein the expression cassette comprises a polynucleotide operably linked to an expression promoter in a sense orientation.

29. The plant of claim 24, wherein the replication cassette is episomal.

30. The plant of claim 29, wherein the replication cassette is a plasmid.

31. The plant of claim 24, wherein the replication cassette is integrated into a eukaryotic chromosome.

32. The plant of claim 24, wherein the replication cassette comprises at least 200 base pairs of DNA that is at least 70% identical to chromosomal DNA in the plant cell.

33. The plant of claim 32, wherein the replication cassette comprises at least 200 base pairs of DNA that is substantially identical to chromosomal DNA in the plant cell.

34. The plant of claim 24, wherein the replication cassette comprises a recombination sequence.

35. The plant of claim 34, wherein the recombination sequence is a lox sequence.

36. The plant of claim 24, further comprising a polynucleotide encoding a sequence-specific recombinase operably linked to a promoter.

37. The plant of claim 36, wherein the sequence-specific recombinase is the Cre recombinase.

38. The plant of claim 24, wherein the eukaryotic replication promoter is tissue-specific.

39. The plant of claim 24, wherein the eukaryotic replication promoter is constitutive.

40. The plant of claim 24, wherein the eukaryotic replication promoter is meiosis-specific.

41. The plant of claim 24, wherein the eukaryotic replication promoter is inducible.

42. The plant of claim 24, wherein the polynucleotide(s) encoding T7 RNA polymerase, T7 gene 4 protein, T7 DNA polymerase and TrxA each encode a nuclear localization signal.

43. A replication system comprising a polynucleotide encoding each of the following polypeptides: T7 RNA polymerase, T7 gene 4 protein, T7 DNA polymerase and TrxA, wherein the polynucleotide encoding each polypeptide is operably linked to a eukaryotic replication promoter.

44. The replication system of claim 43, further comprising a polynucleotide encoding a sequence-specific recombinase.

45. The replication system of claim 44, wherein the sequence-specific recombinase is the Cre recombinase.

46. A polynucleotide, comprising
a bacteriophage T7 origin of replication;
a recombination sequence; and
an expression cassette comprising a eukaryotic replication promoter.

47. The polynucleotide of claim 46, wherein the recombination sequence is a lox sequence.

48. The polynucleotide of claim 46, wherein the polynucleotide comprises a T7 promoter.
